(19) United States
(12) Patent Application Publication
(10) Pub. No.: US 2024/0085409 A1
(43) Pub. Date: Mar. 14, 2024

(54) A reverse lateral flow immunoassay detecting IgE

(71) Applicant: Ke Zhang, Los Angeles, CA (US)

(72) Inventor: Ke Zhang, Los Angeles, CA (US)

(21) Appl. No.: 17/754,730

(22) PCT Filed: Oct. 2, 2020
(86) PCT No.: PCT/US2020/054027

§ 371 Date: Apr. 11, 2022

Related U.S. Application Data
(60) Provisional application No. 62/926,528, filed on Oct. 27, 2019.

(51) Int. Cl. G01N 33/543; G01N 33/558; G01N 33/68

(52) U.S. Cl. CPC G01N 33/54388; G01N 33/558; G01N 33/6854

(57) ABSTRACT

This invention discloses a method and composition for detecting and quantifying the human antigen-specific immunoglobulin E (IgE) by a reverse lateral flow immunoassay. In one embodiment of the invention, the human antigen-specific IgE in in a test sample obtained from bodily fluids including whole blood, dried blood, serum, plasma, saliva, tear, secreting fluid from gastrointestinal tract, respiratory tract and inflammation sites, reacts with nanoparticles-coupled antigens, including allergens, natural environmental antigens, pathogenic antigens, autoantigens, carbohydrate antigens, lipid antigens, synthetic drug antigens and haptens. The formed IgE-antigen-nanoparticle complexes are captured by an anti-human IgE antibody dispensed in the position of the Test line, thus developing a chromatographic color either visualized by naked eye or detectable by a lateral flow reader, and the residual antigen-nanoparticle complex will be captured by anti-antigen specific antibody dispensed in the position of Control line to serve as an assay control. The term of “reverse” specifically indicates that the antigen or antigens are conjugated or coupled to the nanoparticles for this claimed lateral flow immunoassay for human IgE detection and quantification, in conjugation with the capture of the formed antigen specific IgE-antigens-nanoparticles by the immobilized anti-IgE antibody in the Test line position.

### CROSS-REFERENCE TO RELATED APPLICATIONS

This application is based upon and claims the benefit of U.S. Provisional Patent Application No. 62/926,528 filed with the U.S. Patent and Trademark Office on 27 Oct. 2019 and titled “A reverse lateral flow immunoassay method for measurement of antigen-specific IgE”, which application is incorporated herein by reference in its entirety.

### FIELD OF THE INVENTION

The present invention is directed to a method and composition for detecting or quantifying the antigen-specific immunoglobulin E (IgE) in the biological samples by a reverse lateral flow immunoassay using a procedure of coupling the antigens, to which the IgE are specific for and reactive to, to the colored nanoparticles. The formed antigen specific IgE-antigen-nanoparticle complex is then captured and detected by an anti-IgE antibody immobilized on the test line or area of the test strip in the lateral flow immunoassay device.

### BACKGROUND OF THE INVENTION

Over the past decades, numerous patents have been issued involving immuno-chromatographic devices. The standard features of these devices comprise the following:
a. A plastic or paper housing allowing the viewing of a reaction area on a bibulous strip in the form of lateral flow assay;
b. An opening at one end of the housing allowing for the addition of sample (urine, whole blood, plasma, serum, saliva, or other bodily fluids);
c. Bibulous material having immobilized specific binding members capable of reacting with antigens or antibodies;
d. A pad of absorbent bibulous material, e.g., the absorbent pad, enclosed at the end opposite the sample port and used to absorb transversely flowing sample, buffers and colloids;
e. A strip of bibulous material used in the sample port end to initially absorb the sample being applied;
f. A strip of bibulous material in contact with the sample port material and the lateral flow strip and containing a dried colored solid phase reagent, the solid phase coated with antibodies or other proteins.

Two types of chromatographic immunoassays are commonly described, of which in one type, the analytes (proteins, haptens, or small molecules) contained in bodily fluids (urine, whole blood, plasma, serum, and saliva) are detected. The analytes include hCG, FSH, TSH, troponins, myoglobulin, serum proteins, viral or bacterial proteins, haptens, therapeutic drugs, and drugs of abuse.

In the other type of the chromatographic immunoassay, the analyte being detected is (are) human antibody (antibodies) of various classes specifically reactive with agents such as viral or bacterial proteins (HIV, Hepatitis A and C, H. pylori, EBV, Rubella, CMV, HSV, Dengue fever, Lyme, Chagas, TB, Toxoplasma, autoimmune antigens, etc.) or allergens (pollens, molds, dust/mites, foods, animal epithelia, etc.). When it comes to detecting antibody, three formats are typically used:
1) The colored solid phase or nanoparticles are conjugated with proteins or lectins [protein A, protein G, lentil lectin, jacalin, concanavilin A, mannan binding protein, wheat germ lectin, peanut lectin, etc] that react with human IgG antibodies. The solid phase may be coated with anti-immunoglobulins that specifically react with IgG, IgM, and IgA contained in the sample to be analyzed. The bibulous strip would in this case contain the analyte of interest to which the specific antibody contained in the sample reacts.
2) The colored solid phase contains the analyte to which the human immunoglobulins react. The bibulous strip would in this case also contain the analyte of interest to which the specific antibody contained in the sample reacts.
3) The colored solid phase or nanoparticles contains the analyte to which immunoglobulins react. The bibulous strip contains proteins directed against various classes of immunoglobulins or substances such as protein A, protein G, lectins, lentil lectin, jacalin, concanavilin A, mannan binding protein, wheat germ lectin, peanut lectin, or a mix of antibody to immunoglobulin classes IgG, IgA, IgM.

The U.S. Pat. No. 6,528,325 (Hubscher et al) and its later supplementary version U.S. Pat. No. 6,528,325 B1 (Hubscher et al) claimed a lateral flow-based method for detection of antigen-specific IgM, IgG and IgA using the approach of coupling the antigens to the chromatographic particles, the IgM-, IgG- and IgA-containing immunocomplex were then captured by the anti-IgM, anti-IgG and anti-IgA antibodies dispensed in the test line of the nitrocellulose membrane. In another embodiment of the same patent, the inventors also claimed a method to detect allergen-specific IgE using anti-IgE antibody-coupled chromatographic particles. In this IgE detecting method, the allergen (s) were immobilized in the test line of the bibulous nitrocellulose membrane to detect the IgE-containing complex. Such an anti-IgE antibody conjugated particle-based allergen-specific IgE detecting method is thus referred as “conventional lateral flow Immunoassay” hereafter.

U.S. Pat. No. 7,629,127 (Hubscher) disclosed an improved version of a lateral flow immunoassay described in the U.S. Pat. No. 6,528,325 (Hubscher et al) to enhance the reading and detecting sensitivity for allergen specific IgE detection using a “two-step” approaching by designing a buffer port upstream the sample port, and applying a chase buffer in this buffer port following the sample application in the sample port. In this method, the anti-IgE antibody labelled conjugates were dried in the position between buffer port and sample port. In this two-step” alternative form of the conventional LFIA, the allergens were immobilized in the test area of the strip.

An alternative form of the “conventional lateral flow immunoassay” for IgE detection is the European patent EP1891447A1 (Rundstrom et al), wherein a two-step approach was applied for improved detection of allergen-specific IgE. In this two-step” assay, the IgE-containing sample was first applied to the sample port and let it flow towards to the wicking pad, followed by applying the running buffer in the buffer port that was positioned upstream of the sample port. The dried anti-IgE antibody conjugates were deposited in a position between the buffer port and sample port. In this two-step” alternative form of the conventional LFIA, the allergens were immobilized in the test area of the strip.

Due to sensitivity and/or specificity limit, the prior invention of “conventional lateral flow immunoassay” for allergen-specific IgE detection could not be widely applied for allergy diagnoses, particularly in food allergy diagnosis. It would be desirable to having a highly sensitive and specific lateral flow-based assay that can overcome the low sensitivity problem in the prior art for detecting and quantifying the allergen-specific IgE in biological samples as a rapid screening and diagnostic tool to diagnose IgE-mediated allergies, including food allergy.

### SUMMARY OF THE INVENTION

This invention is about a novel reverse lateral flow immunoassay (R-LFIA) platform capable of highly sensitively and specifically detecting allergen- and antigen-specific IgE in a rapid manner and a point-of-care setting as a diagnostic tool to diagnose IgE-mediated diseases including allergic and autoimmune diseases.

It is, therefore, one of the objects of the present invention is a novel R-LFIA platform capable of detecting soluble IgE, particularly human IgE with specificity to various antigens and allergens, including but not limit to, food allergens, autoantigens, environmental allergens, insect antigens, parasite antigens, bacterial and virus antigens and synthetic drug antigens or heptens that are responsible for IgE-mediated allergic diseases, autoimmune diseases and other IgE-mediated diseases.

It is, therefore, another one of the objects of the present invention to enable the diagnosis of food allergy such as peanut, milk, egg, shellfish, fish, tree nuts, red meat, wheat, soys, sesame seeds; and IgE-mediated diseases against the autoantigens in a form of soluble proteins, membrane proteins, intracellular proteins, protein-DNA complexes, protein-RNA complexes that are served as the autoantigens responsible for the autoimmune based mast cells and basophil activation such as chronical spontaneous urticaria and other idiopathic acute and chronical urticaria; environmental allergens, such as dust mite, cockroach, pollen, pet dander, mold; IgE against insect allergens such as venom of bees, wasps, hornets, yellow-jackets and fire ants; IgE against parasitic antigens such as helminths, hookworms and other parasitic worms; and IgE against the synthetic drugs, antibiotics and anti-tumor drugs.

It is another one of the objects of the present invention to provide a novel reverse lateral flow immunoassay assay, including a test strip and/or a cassette for holding the test strip, that can detect one or more allergen- or antigen-specific IgE in a sample qualitatively, semi-quantitatively, and quantitatively, in a point-of-care setting, and in a rapid manner.

### BRIEF DESCRIPTION OF THE DRAWINGS

FIG. 1 is a diagram of an example of a test trip. FIG. 1A is the test strip without separate sample pad, and FIG. 1B is the one with separate sample pad as an alternative setting format.

FIG. 2 is the mechanistic diagram of the novel reverse lateral flow immunoassay. FIG. 2A is the diagram for positive allergen specific IgE detection, using peanut allergen specific IgE as an example. FIG. 2B is the diagram for negative allergen specific IgE detection, using peanut allergen specific IgE as an example. FIG. 2C is a real strip example demonstrating the peanut positive and negative results detected by R-LFIA.

FIG. 3 is a comparison of the reverse lateral flow immunoassay (R-LFIA) with the conventional lateral flow immunoassay (C-LFIA). FIG. 3A is the mechanistic diagram for the R-LFIA, and FIG. 3B is the mechanistic diagram for the C-LFIA. The corresponding components in both R-LFIA and C-LFIA are individually labelled and indicated in FIG. 3.

FIG. 4 is the example of detecting sensitivity comparison between R-LFIA and C-LFIA using the same peanut allergic plasma sample. The asterisk represents the ending dilutions to be detected with the respective methods.

FIG. 5 is the example of ending dilutions detected by R-LFIA from three peanut allergic plasma samples. The corresponding international Unit (IU) level measured with ImmunoCAP method is used for comparison.

FIG. 6 is the example of R-LFIA detection results using various volume applied.

FIG. 7 is the example of the representative R-LFIA detection results from the random normal population without known peanut allergy.

FIG. 8 is the example of specificity of the peanut allergic IgE detecting R-LFIA, which specifically detects the peanut allergic IgE but not IgE specific to other allergens.

FIG. 9 is the example of the R-LFIA test results for the Basophil Activation Test confirmed peanut allergic serum samples.

FIG. 10 is the summary of the test results of the peanut non-allergic and allergic samples for preliminary diagnostic cutoff value determination.

FIG. 11 is the example of the R-LFIA test results of 28 plasma samples with peanut allergic IgE value higher than the class 4 of the ImmunoCAP method classification.

FIG. 12 is the example of ending dilutions detected by R-LFIA from three shrimp allergic plasma samples. The corresponding IU level measured with ImmunoCAP method is used for comparison.

FIG. 13 is the example of the R-LFIA test results of 19 plasma samples with shrimp allergic IgE value higher than the class 4 of the ImmunoCAP method classification.

### DETAILED DESCRIPTION OF THE INVENTION

The invention may be understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

More than 32 million Americans suffer from food allergies, for example, peanut allergy. Some of them unnecessarily given the high false positive rate of current diagnostics. Thus, the oral food challenge (OFC) remains the gold standard for a definitive diagnosis of food allergy. However, because OFC is a high-risk resource-intensive procedure needing close supervision in well-equipped medical facility, is not routinely accessible to a large portion of food allergy patients. Many are left without a confirmed diagnosis until the allergic reaction accidently occurs. It is unequivocally agreed that accurate food allergy diagnostics are urgent medical needs.

Flow Immunoassay has been widely used as an inexpensive rapid diagnostic suitable for Point-Of-Care (POC) settings for various diseases, including environmental allergies. However, the current C-LFIA format cannot achieve sufficiently high sensitivity to serve as a meaningful diagnostic for many allergies. Given the limitations of the high false positivity of all available commercial allergen specific-IgE tests and the low sensitivity of C-LFIA, this invention of a R-LFIA for allergen specific IgE detection is designed to mitigate these problems.

The R-LFIA format for peanut allergic IgE test not only displays high specificity by filtering out low affinity, cross-reactive IgE but also significantly enhances the IgE detection sensitivity by ˜30 fold compared to that of the C-LFIA format, thus reaching the threshold for clinically application for peanut allergy diagnosis.

The R-LFIA format also is applicable for other food allergic IgE and other environmental allergen and autoantigen specific IgE test with the same or similar high sensitivity and specificity.

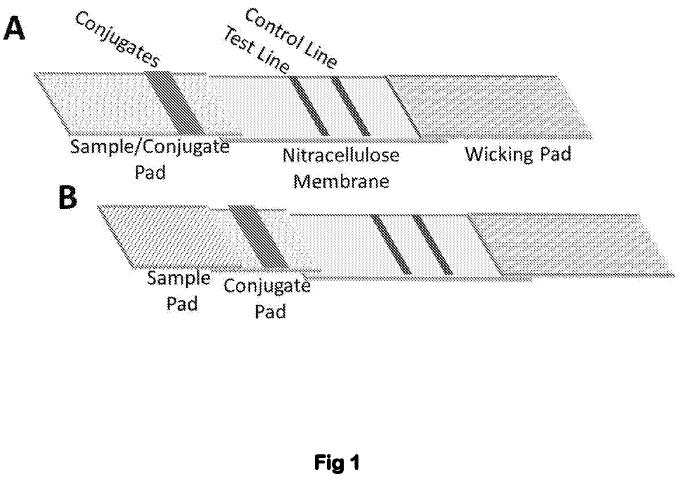

The said R-LFIA is a test strip comprised the following components, with following characteristics:
1. A sample pad.
2. A conjugate pad.
3. A combined pad serving the purpose of both sample pad and conjugate pad as an alternate (FIG. 1A).
4. Conjugates containing gold nanoparticles coupled with antigens or allergens that are dried down to the conjugate pad.
5. A protein-binding membrane pre-dispensed with the anti-human IgE antibody at the test line position, and an anti-antigen antibody pre-dispensed in the position of the control line.
6. An absorbent pad.
7. All the above components are mounted onto a backing supporter.
8. There are overlaps between the sample pad and the conjugate pad, between the conjugate pad and the protein-binding membrane, and between the protein-binding membrane and the absorbent pad.

The said test procedures for the said reverse lateral flow immunoassay comprise the following steps:
1. Directly apply the testing sample, diluted or non-diluted, in a volume of 0.1 uL to 1 uL, or in a volume of 1 uL to 50 uL, with 0.1 uL interval, to the sample port.
2. Following the sample addition to the sample port, a drop of running buffer could be immediately, or waiting for a designed time frame that could be 1″ to 60″, and 60″ to 120″, with 1″ interval, onto the sample port.
3. Alternatively, the various volume of samples to be tested, diluted or non-diluted, could be premixed with running buffer, then applying to the sample port of the lateral flow device.
4. The test results, indicated by color density, can be visualized between 5 second and 10 minutes, or longer than 10 minutes, depending on the concentration of the analytes in the sample.
5. The color density can be visualized or read by a designed lateral flow reader.

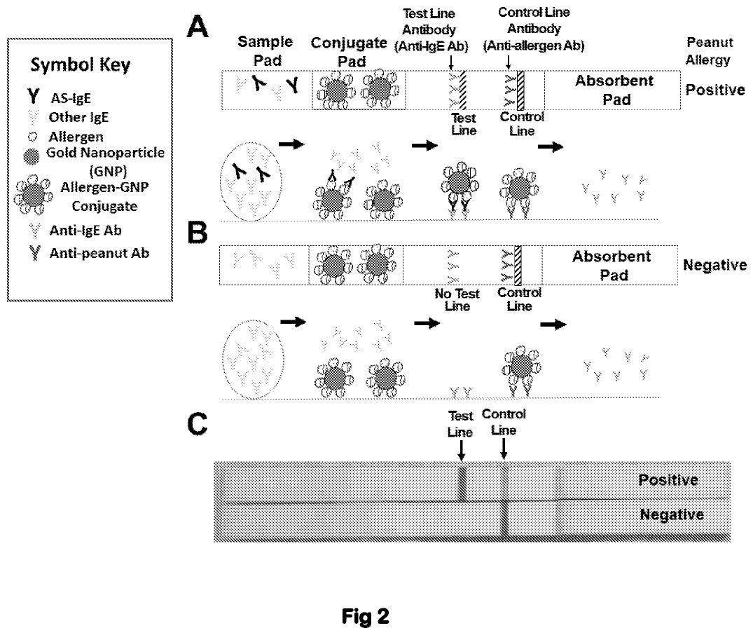

The antigen (hereafter using peanut allergen as an example) specific-IgE in testing samples binds to the allergens pre-conjugated to the Gold Nano-Particle (GNP) that was dried down to the conjugate pad, leaving other IgE remaining unbound (FIG. 2). The peanut specific IgE-peanut allergen-GNP complex, when flowing through toward absorbent pad, is captured by an anti-IgE antibody (Ab) printed on the position of test line, forming the visible/detectable Test Line signal. The IgE-free Peanut allergen-GNP conjugate passes over the test line but captured by the Anti-peanut MAb printed on the control line, forming the Control Line signal (FIG. 2A). In the case of non-peanut allergy, no peanut specific IgE is available to form a complex with peanut allergen-GNP for Anti-IgE Ab to capture on test line, thus no Test Line signal will be visible/detectable. However, the Peanut allergen-GNP conjugate would be captured by the Anti-peanut MAb to form a Control Line (FIG. 2B). A real test example of peanut positive and negative samples is displayed in FIG. 2C.

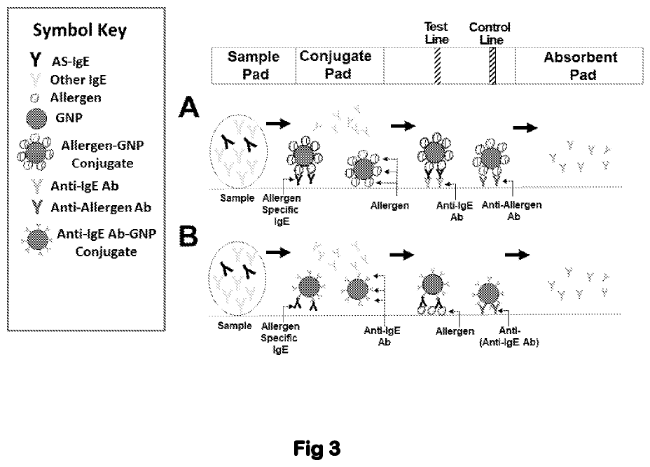

In R-LFIA format, the allergen-specific IgE, as well as other Ig isotypes, would directly compete to bind to the allergen coupled to GNP (FIG. 3A). The allergen specific IgE-containing GNPs are captured by the anti-IgE antibodies immobilized on the test line, whereas the GNP not containing the allergen specific IgE would pass through the test line and subsequently captured by an anti-peanut MAb immobilized in the control line. Hence the none-allergen specific IgE (e. g., other IgE) level in the sample theoretically would compete with allergen specific IgE for binding to test line. However, such a possibility is not an issue in LFIA setting as the immobilized anti-IgE Ab amount are extremely excessive so that the serum IgE level would not be able to saturate all the IgE binding capacity in test line.

Comparison with conventional lateral flow immunoassay (C-LFIA). For C-LFIA (FIG. 3B), the anti-IgE antibodies are conjugated to the GNP. The total IgE, as well as the allergen specific IgE, from plasma are captured by anti-IgE MAb coupled to gold nanoparticles (anti-IgE-Gold conjugate). When the gold nanoparticle-containing complexes flow over the test line where the allergens (hence peanut allergens) are coated, only the complexes carried allergen specific IgE would be captured by coated allergen and therefore deposited as the visible (or detectable) signal, as the allergen specific IgE acts as a bridge to immobilize the complexes at the test line, whereas the gold nanoparticle complexes carrying total IgE other than allergen specific IgE, the complexes would pass through the test line, and captured by anti-(anti-IgE) MAb coated at the control line. In this format, the total IgE/allergen specific IgE ratio level would be a key variable impacting the assay sensitivity, as the total IgE level in the sample would directly compete allergen specific IgE for binding to the anti-IgE coupled to gold nanoparticles. In this format, the allergen specific Ig (IgM, IgG and IgA) level is irrelevant, as they would not specifically bind to GNP to influence the assay sensitivity.

### Examples

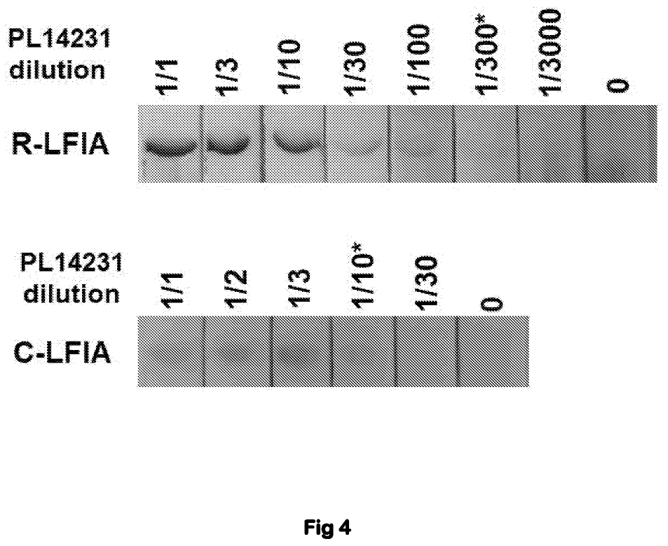

Example 1. Detection sensitivity comparison between R-LFIA and C-LFIA using the same peanut allergic sample (PL14231). The asterisk represents the ending dilutions to be detected with the respective methods (FIG. 4). With optimal dotting conditions, the R-LFIA was able to pick up a positive signal from 1:300 dilution, whereas the C-LFIA was capable of detecting a positive signal at 1:10 dilution, indicating a 30-fold higher sensitivity of R-LFIA than that of C-LFIA for peanut specific IgE detection.

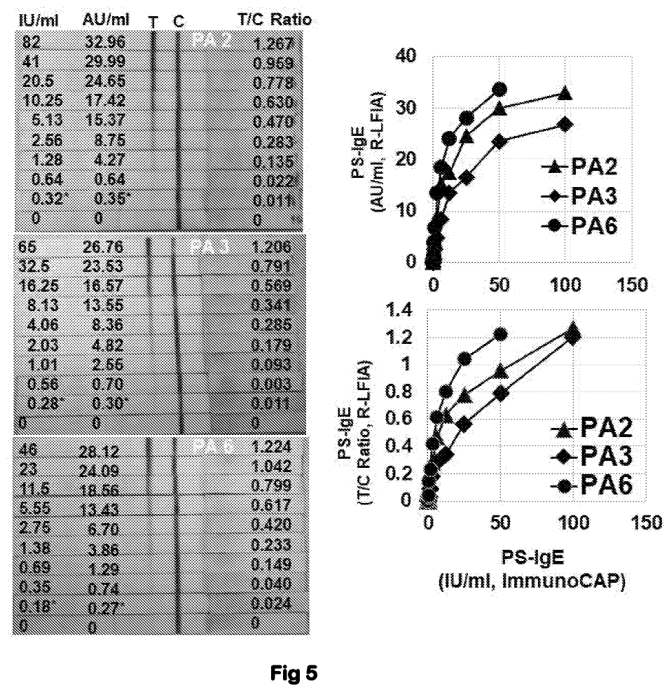

Example 2. The ending dilutions detected by R-LFIA from three (PA2, PA3 and PA6) peanut allergic plasma samples. The corresponding IU level measured with ImmunoCAP method is used for comparison to determine the R-LFIA sensitivity limit (FIG. 5). A series 2-fold dilution test revealed that R-LFIA was capable of picking up the signal level equivalent or lower than that of the sensitivity limit for ImmunoCAP (0.35 IU/mL, or 0.35 kU_{A}/L), indicating equivalent or higher sensitivity than that of the ImmunoCAP IgE test.

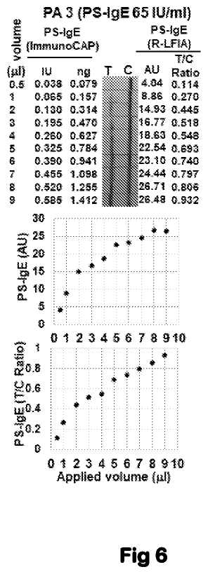

Example 3. R-LFIA detection results using various volume of a peanut allergic plasma. As low as 0.5 uL of PA3 sample sufficiently resulted in visible positive signal measured with R-LFIA (FIG. 6). The incremental sample volume escalation from 0.5 uL to 9 uL accordingly increased the positive signal level almost linearly, particularly when the AU value was normalized. These data demonstrated that R-LFIA is a highly sensitive as well as a practically flexible PA diagnostic tool capable of accommodating a wide range of sample volumes for diagnostic testing.

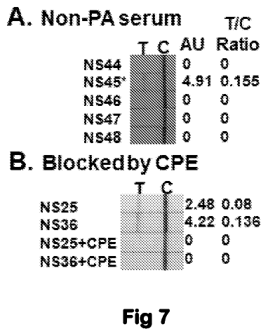

Example 4. The peanut specific IgE level in random normal population without known peanut allergy. While 90% random normal population did not show any detectable peanut specific IgE, about 10% did display weak positivity with AU<6.0 (For example, the samples NS45, FIG. 7). These weakly positive samples could be completely inhibited by 1 uL of 100 ug/mL Crude Peanut Extract, indicating that R-LFIA picked up low level peanut specific IgE from the random population.

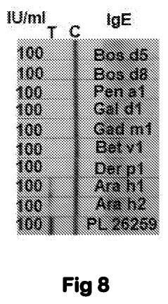

Example 5. Specificity of the R-LFIA for peanut specific IgE detection. The peanut IgE R-LFIA was only reactive with the recombinant IgE specific for Ara h1 and Ara h2, but not cross-reactive with 100 IU/ml allergen specific IgE to milk (Bos d5 & Bos d8), shrimp (Pen a1), egg (Gal d1), fish (Gad m1), birch pollen (Bet v1), and house mite (Der p1). As a positive control, the PA sample PL 26259 displayed strongly positive reactivity (FIG. 8).

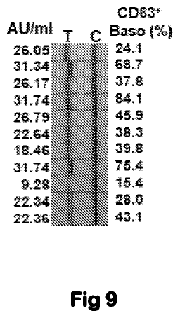

Example 6. Correlation of R-LFIA IgE test results with that of the Basophil Activation Test (BAT). All 11 BAT-confirmed peanut allergic samples exhibited high AU levels using peanut IgE R-LFIA (FIG. 9).

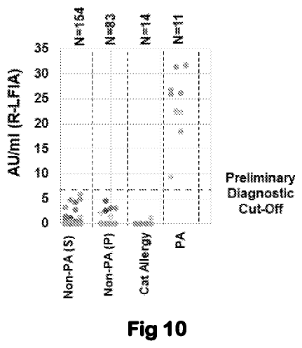

Example 7. Diagnostic cutoff value of the peanut IgE R-LFIA. With the data available from this pilot study, if a cutoff value is set at AU=6, then the R-LFIA displays 100% (11/11) positive predictive value, and 100% (251/251) negative predictive value for peanut allergy diagnosis (FIG. 10).

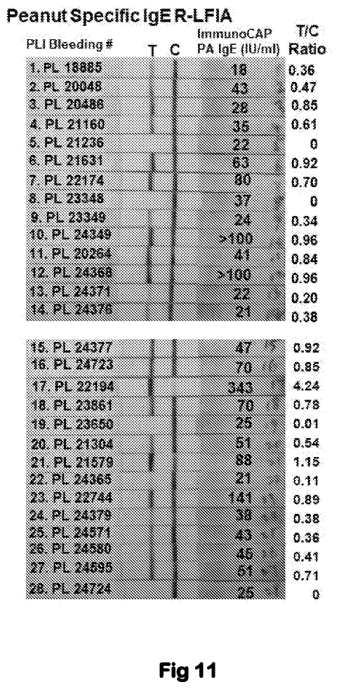

Example 8. R-LFIA IgE test results for the plasma samples with peanut specific IgE level>17.5 IU/mL (Class IV and above of ImmunoCAP classification) (FIG. 11). These data collectively show that 21.5% (6 of 28) of samples with class IV level of PS-IgE determined by ImmunoCAP contained no or very low AU measured with R-LFIA.

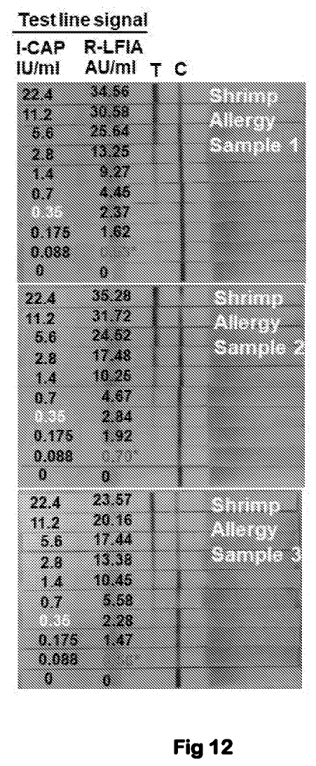

Example 9. The ending dilutions detected by R-LFIA from three shrimp allergic plasma samples. The corresponding IU level measured with ImmunoCAP method is used for comparison to determine the R-LFIA sensitivity limit (FIG. 12). A series 2-fold dilution test revealed that R-LFIA was capable of picking up the signal level equivalent or lower than that of the sensitivity limit for ImmunoCAP (0.35 IU/mL, or 0.35 kU_{A}/L), indicating equivalent or higher sensitivity than that of the ImmunoCAP IgE test.

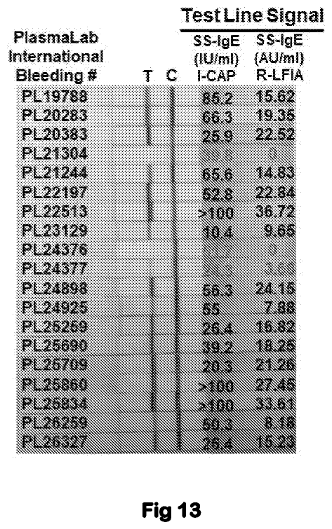

Example 10. R-LFIA IgE test results for the plasma samples with shrimp specific IgE level>17.5 IU/mL, which are Class IV and above of ImmunoCAP classification (FIG. 13). These data collectively show that 15.8% (3 of 19) of samples with class IV level of PS-IgE determined by ImmunoCAP contained no or very low AU measured with R-LFIA.

What is claimed is:

1. A reverse lateral flow immunoassay is a method specifically designed for detecting the human antigen-specific immunoglobulin E (IgE). The said reverse lateral flow immunoassay comprises following components and procedures.
a. The said reverse lateral flow immunoassay method is a test strip comprised of a sample pad; a conjugate pad, or a combined pad serving the purpose of both sample pad and conjugate pad as an alternate; a protein-binding membrane; and an absorbent pad, mounted onto a backing supporter. There are overlaps between the sample pad and the conjugate pad, between the conjugate pad and the protein-binding membrane, and between the protein-binding membrane and the absorbent pad.
b. The conjugates are pre-dispensed and dried down to the conjugate pad. Alternatively, the conjugates can be in liquid form mixing with samples to be tested, then applying to the sample port of the lateral flow device.
c. Anti-human IgE antibodies are pre-dispensed in the position of Test Line on the protein-binding membrane.
d. The anti-antigen (e.g. anti-peanut) antibodies are pre-dispensed in the position of Control Line on the protein-binding membrane.
e. The test strip can be housed in a cassette, or in a dipstick form.
f. The test procedure is to directly apply the sample to the sample pad, or the conjugate pad in the case of combined sample pad and conjugate pad, followed by applying a running buffer onto the sample/conjugate pad immediately following the sample addition, or with a predefined time interval. Alternatively, the samples to be tested can be premixed with running buffer, then applying to the sample port of the lateral flow device.
g. The test results can be visualized or machine-read by a designed lateral flow reader.

2. The said reverse lateral flow immunoassay method of claim 1, wherein said lateral flow immunoassay method is a test strip either housed in a cassette, or in a dipstick form.

3. The said reverse lateral flow immunoassay method of claim 1, wherein said “reverse” specifically refers the procedures of coupling or conjugating of antigens, directly or indirectly, chemically or physically, to the colorimetric nanoparticles.

4. The said reverse lateral flow immunoassay method of claim 1, wherein said conjugate specifically refers to the complexes with antigens conjugated to the colorimetric nanoparticles.

5. The said reverse lateral flow immunoassay method of claim 1, wherein said colorimetric nanoparticles include, but not limit to, colloid gold nanoparticles, silver nanoparticles, latex nanoparticles, magnetic nanoparticles, fluorescent labelled nanoparticles.

6. The said reverse lateral flow immunoassay method of claim 1, wherein said protein-binding membrane is the solid supporter capable of immobilizing and bind proteins and antibodies, including but not limit to, nitrocellulose membrane, and polyvinylidene difluoride (PVDF) membrane.

7. The said reverse lateral flow immunoassay method of claim 1, wherein said antigens including, but not limit to, allergens, natural environmental antigens, pathogenic antigens, autoantigens, carbohydrate antigens, lipid antigens, synthetic drug antigens and haptens.

8. The said reverse lateral flow immunoassay method of claim 7, wherein said allergens including, but not limit to, food allergens such as peanut, cow's milk, egg white, shellfish, crustaceans, fish, tree nuts, red meat, wheat, soy, sesame seeds; environment allergens such as dust mite, pollen, cockroach, pet dander, mold; insect allergens such as venom of bees, wasps, hornets, yellow jackets and fire ants, allergen mixes, and combinations thereof.

9. The said reverse lateral flow immunoassay method of claim 7, wherein said autoantigens including, but not limit to, the soluble proteins from humans, the cell surface membrane proteins, intracellular proteins, DNA, RNA and the complexes of DNA/RNA and proteins.

10. The said reverse lateral flow immunoassay method of claim 7, wherein said pathogenic antigens include, but not limit to, parasitic antigen such as helminths and other parasitic worms, bacterial antigens, fugus antigens, virus antigens such as the novel coronavirus 2019 (SARS-CoV-2) spike and nucleocapsid proteins.

11. The said reverse lateral flow immunoassay method of claim 7, wherein said synthetic drug antigens and haptens including, but not limit to, antibiotics, anti-fugus drugs, anti-virus drugs, anti-tumor drugs, and other synthetic small molecules and haptens.

12. The said reverse lateral flow immunoassay method of claim 1, wherein said anti-human IgE antibodies including, but not limit to, polyclonal or monoclonal antibodies to human IgE Fc region derived from mouse, rabbit, rat, hamster, horse, sheep, goat, chicken, camelids, llama, cartilaginous fish, humans, humanized, recombinant.

13. The said reverse lateral flow immunoassay method of claim 1, wherein said anti-antigen antibodies including, but not limit to, polyclonal or monoclonal antibodies to antigen derived from mouse, rabbit, rat, camelid, humans, humanized, recombinant.
